(19) United States
(12) Patent Application Publication
(10) Pub. No.: US 2010/0047391 A1
(43) Pub. Date: Feb. 25, 2010

(54) REDUCED-SALT DAIRY PRODUCT WITH IMPROVED TASTE

(76) Inventors: Frank Richard Meijer, Rotterdam (NL); Catharina Houdijk, Rotterdam (NL); Cornelis Muijlwijk, Dalfsen (NL); Neeltje Corine Oorschot Van, Haarlem (NL)

Correspondence Address: NIXON & VANDERHYE, PC, 901 NORTH GLEBE ROAD, 11TH FLOOR, ARLINGTON, VA, 22203, US

(21) Appl. No.: 12/519,561

(22) PCT Filed: Dec. 17, 2007
(86) PCT No.: PCT/EP07/64058

§ 371 Date: Aug. 3, 2009

(30) Foreign Application Priority Data
Dec. 19, 2006 (EP) 06123506.2
Jun. 4, 2007 (EP) 07109506.1
Jul. 25, 2007 (EP) 07113092.6

(51) Int. Cl. A23C 15/12; A23C 9/12; A23C 19/04; A23L 1/22; A23L 1/307; C07H 19/167; C07H 19/067

(52) U.S. Cl. 426/37; 426/42; 426/580; 426/582; 426/581; 426/548; 536/27.2; 536/28.4

(57) ABSTRACT

The present invention relates to a method for preparing a dairy product with reduced sodium, wherein a yeast extract comprising at least 30% w/w 5′-ribonucleotides, on the basis of sodium chloride free dry matter, is added to a dairy composition used in the production of dairy product with reduced sodium. Preferably, a yeast extract is used wherein the total amount of 5′-GMP plus 5′-IMP is at least 15% w/w on the basis of sodium chloride free dry matter. The method of the invention may advantageously be used since off-tastes are masked and salt perception is enhanced.

### FIELD OF THE INVENTION

The present invention relates to a method for producing a dairy product with reduced sodium content. The invention further relates to a dairy product with reduced sodium content. In particular, it relates to the improvement of taste of dairy products with reduced sodium content.

### BACKGROUND OF THE INVENTION

The use of sodium chloride has long been suspected as a cause of health problems, particularly hypertension. It is very difficult to limit salt intake to very low levels because processed foods, takeaway foods and restaurant-prepared foods typically contain large amounts of salt. At the same time, there is a trend with many food processors to reduce the amount of sodium in processed food. To some degree the problem of high salt levels in foods, such as dairy, could be reduced by simply lowering the level of salt added to the food, however this leads to a reduced salt perception and is therefore undesired. A possible solution thereto could be the use of salt substitutes. However a disadvantage of salt substitutes, such as potassium chloride, is that they impart an “off” flavor to food. As a consequence, it is necessary to use additives to mask the off-flavor of the salt substitutes.

U.S. Pat. No. 4,297,375 discloses the use of an autolyzed yeast to reduce bitterness in foods caused by the presence of potassium chloride-containing salt substitutes.

EP0103994 describes a sodium chloride substitute flavoring system which comprises autolyzed yeast and ammonium chloride. The system improves the saltiness of foods, reduces the bitterness of sodium chloride substitutes and is suitable for use in a sodium chloride substitute system for savoury applications.

WO 2005/087013 describes the use of a dry taste enhancing agent for cereal products which taste-enhancing agent comprises a yeast extract and acid fermented flour.

A problem associated with autolysed yeast but also with yeast extracts as generally described in the prior art is that they can cause the food to which they are added to get a bouillon-like, brothy taste or after taste associated with yeast (yeasty taste and/or odour). This problem is especially evident in beverages and in non-savoury food.

Object of the present invention is to provide a method to produce a dairy product with reduced sodium content and to provide a dairy product with reduced sodium content obtainable therefrom wherein the salt perception in the dairy product should be improved. Furthermore the off-flavour or bitterness caused by traditional salt replacers optionally present in the dairy product should be masked without imparting to the food a bouillon-like, brothy taste.

### DETAILED DESCRIPTION OF THE INVENTION

The present invention relates to a method for preparing a dairy product with reduced sodium content in comparison to a corresponding dairy product, wherein a yeast extract comprising at least 30% w/w 5′-ribonucleotides, on the basis of sodium chloride free dry matter, is added to a dairy composition used in the production of a dairy product with reduced sodium content in comparison to a corresponding dairy product.

There are several advantages of using such yeast extract for preparing a product with reduced sodium content in comparison to a corresponding dairy product. One advantage is that bitter off-tastes, for instance from salt substitutes such as potassium chloride, are masked if a yeast extract comprising at least 30% w/w 5′-ribonucleotides on the basis of sodium chloride free dry matter is added to a dairy composition comprising salt substitutes used in a dairy production process.

Another advantage is that the yeast extract effects a neutral taste enhancement, i.e. taste enhancement without the introduction of a brothy, bouillon note or yeasty note.

Yet another advantage is that by using a yeast extract which comprises at least 30% w/w 5′-ribonucleotides on the basis of sodium chloride free dry matter the salt perception in the dairy product is enhanced. This is of course very advantageous in food applications with reduced sodium content. As a result, less sodium and less sodium replacers or taste enhancers are necessary to improve the taste of a dairy product with reduced sodium content.

Another advantage is that there is no requirement to label the dairy products which have been prepared according to the invention as containing chemical additives.

In the context of the present invention a ‘dairy product’ refers to any kind of milk-based product, including but not limited to cheese, butter, ice cream, casein, dairy-based meat replacers, yogurt and yogurt-based products, such as tzatziki, their analogues or replacements. Of particular interest in the present context are cheese and milk fat containing products and their equivalents, including regular cheeses, cheese analogues, processed cheeses, butter, spreads and margarines, be it in a full fat, reduced fat or low-fat formulation.

In a preferred embodiment, the dairy product is a cheese. The cheese may be of any variety, e.g. hard cheeses such as Chester, Danbo, Manchego, Saint Paulin, Cheddar, Monterey, Colby, Edam, Gouda, Muenster, Swiss type, Gruyere, Emmenthaler, Parmesan and Romano; pasta filata cheeses such as Mozzarella; processed cheese; white mould cheese such as Brie and Camembert; or blue mould cheeses such as Gorgonzola and Danish blue cheese, or fresh cheese such as e.g. Ricotta, Cream cheese, Neufchatel or Cottage cheese. In one embodiment of the invention the cheese product with reduced sodium is cream cheese with reduced sodium.

Processed cheese is preferably manufactured from regular cheese or cheese analogues by cooking and emulsifying the cheese, such as with emulsifying salts (e.g. phosphates and citrate). The process may further include the addition of spices or condiments.

The term ‘dairy analogues’ refers to dairy-like products which contain fat (such as e.g. milk fat (e.g. cream) as part of the composition, and which further contain, as part of the composition, a non-milk constituent, such as e.g. vegetable oil.

The term “dairy product with reduced sodium content” refers to a dairy product as specified above wherein the amount of sodium in the product is less than the amount of sodium conventionally present in the corresponding dairy product. Preferably the dairy product will comprise at least 25% w/w less sodium than conventionally present in the corresponding dairy product. Said dairy product with reduced amount of sodium may comprise at least 25% w/w, 30% w/w, 35% w/w or 40% w/w less sodium, more preferably at least 45% w/w, 50% w/w, 55% w/w or 60% w/w less sodium than conventionally present in the corresponding dairy product. The dairy product with reduced sodium content prepared according to the method of the first aspect may comprise up to ˜100% w/w less sodium than conventionally present in the corresponding dairy product.

For the sake of clarity, this also covers dairy products in which the sodium of other sodium sources than sodium chloride is replaced. For example, a processed cheese with reduced sodium may contain less sodium chloride or less sodium-containing melting salts, typically, sodium citrate and sodium phosphates, or both. In one embodiment, the yeast extract of the invention is used to enhance the taste of a processed cheese in which the sodium of the melting salts is replaced. Both this type of processed cheese and the method to make this type of processed cheese are encompassed by the present invention.

The amount of sodium conventionally present in the corresponding dairy product is the amount present when the dairy product has not been altered, processed or formulated in order to reduce its sodium content and will depend on the type of dairy product, hence the reference to a ‘corresponding’ dairy product. Those skilled in the art know the amount of sodium which is conventionally present in a dairy product and know that it will depend on the type of dairy product. For example, the corresponding product of a sodium reduced cottage cheese is a cottage cheese which has not been altered, processed or formulated in order to reduce its sodium content. The corresponding product of a sodium reduced processed cheese is a processed cheese which has not been altered, processed or formulated in order to reduce its sodium content. The corresponding product of a sodium reduced cream cheese is a cream cheese which has not been altered, processed or formulated in order to reduce its sodium content. The corresponding product of a sodium reduced low fat spread is low fat spread which has not been altered, processed or formulated in order to reduce its sodium content. The corresponding product of a sodium reduced margarine is margarine which has not been altered, processed or formulated in order to reduce its sodium content.

According to the invention, a yeast extract is used which comprises at least 30% w/w 5′-ribonucleotides, preferably at least 34% w/w, 38% w/w, 40% w/w or 42% w/w, more preferably at least 44% w/w, 46% w/w, 48% w/w or at least 50% w/w 5′-ribonucleotides on the basis of sodium chloride free dry matter. Generally the yeast extract may comprise an amount of 5′-ribonucleotides up to 90% w/w based on sodium chloride free dry matter.

In the context of the present invention, the phrase “5′-ribonucleotides” refers to the total amount of 5′-ribonucleotides formed during RNA degradation, viz. 5′-monophosphate guanine (5′-GMP), 5′-monophosphate uracil (5′-UMP), 5′-monophosphate cytosine (5′-CMP), 5′-monophosphate adenine (5′-AMP), where 5′-AMP may be partially or completely converted into 5′-monophosphate inosine (5′-IMP). For example, in a yeast extract which comprises 30% w/w 5′-ribonucleotides on the basis of sodium chloride free dry matter, the total amount of 5′-GMP, 5′-UMP, 5′-CMP, 5′-AMP and 5′-IMP is 30% w/w on the basis of sodium chloride free dry matter.

In a preferred embodiment, a yeast extract is used wherein the total amount of 5′-GMP plus 5′-IMP is at least 15% w/w, preferably at least 17% w/w, 19% w/w, 20% w/w or 21% w/w, more preferably at least 22% w/w, 23% w/w, 24% w/w or 25% w/w, on the basis of sodium chloride free dry matter. Generally the yeast extract may comprise a total amount of 5′-GMP plus 5′-IMP up to 45% w/w based on sodium chloride free dry matter. Due to the constitution of RNA, from which the 5′-ribonucleotides in the yeast extract arise, 5′-GMP and 5′-IMP will always be present in approximately equal amounts in this embodiment, if substantially all 5′-AMP is converted into 5′-IMP.

In the context of the present invention, weight percentage calculations of the 5′-ribonucleotides are based on the disodium salt heptahydrate thereof unless otherwise specified. All percentages are calculated on sodium chloride free dry matter. In the present invention, the phrase ‘sodium chloride free dry matter’ refers to the fact that for the calculation of the weight percentage the weight of any sodium chloride present is excluded from the composition. The measurement of sodium chloride in the composition and the above-mentioned calculation can be performed by methods known to those skilled in the art.

The yeast extract may be prepared by any method which yields a yeast extract which comprises at least 30% w/w 5′-ribonucleotides on the basis of sodium chloride free dry matter.

In one embodiment of the invention, the yeast extract is obtained by hydrolysis. Hydrolytic yeast extracts are concentrates of the soluble material obtained from yeast after disruption of the cells, digestion (lysis) and addition of exogenous enzymes such as proteases and/or peptidases and especially nucleases, such as 5′-phosphodiesterase and optionally 5′-adenylic deaminase, to the yeast suspension during lysis. The native yeast enzymes are generally inactivated prior to the lysis. During this process 5′-GMP, 5′-UMP, 5′-CMP and 5′-AMP may be formed. When adenylic deaminase is added to the mixture, 5′-AMP is transformed into 5′-IMP. Methods to produce hydrolytic yeast extracts are known in the art, see for example WO88/05267.

In another embodiment, the yeast extract is obtained by autolysis, for instance as described in WO2005/067734.

Preferably, the 5′-ribonucleotides of the yeast extract are produced in an autolytic or hydrolytic extraction process, i.e. in a natural process, as described in the previous paragraph. However, the skilled person will understand that a yeast extract which comprises at least 30% w/w 5′-ribonucleotides on the basis of sodium chloride free dry matter may be obtained by adding isolated or chemically synthesized 5′-ribonucleotides to a yeast extract which contains less than 30% w/w 5′-ribonucleotides. The use of these types of yeast extract is also encompassed in the present invention, but is less preferred. It has the disadvantage that food products prepared with such yeast extracts will have to be labeled as containing chemical additives.

With the methods mentioned above to produce yeast extracts used in the method of the invention also yeast extracts may be produced which do not fulfil the requirements of the first aspect of the invention. However the yeast extracts comprising the proper amount of 5′-ribonucleotides may be selected by measuring the amounts of 5′-ribonucleotides using methods known to those skilled in the art and by selecting those yeast extracts which comprise at least 30% w/w 5′-ribonucleotides on the basis of sodium chloride free dry matter.

The yeast extracts used in the context of the invention may be obtained from any yeast. Suitable yeast strains which may be used belong to the genera Saccharomyces, Kluyveromyces and Candida. Yeast strains belonging to the genus Saccharomyces, for example to the species Saccharomyces cerevisiae, are preferred.

In the context of the present invention, a diary composition may be a composition comprising milk and/or one or more milk components and/or milk fractions which is the starting composition in the production of dairy product, it may be an intermediate product in the production of the dairy product (e.g. curd or whey) or it may be a dairy product. A milk component may be any constituent of milk such as milk fat, milk protein, casein, whey protein, lactose. A milk fraction may be any fraction of milk such as e.g. skimmed milk, butter milk, whey, cream, butter, milk treated by ultrafiltration, milk powder, whole milk powder, butter milk powder, or skimmed milk powder. In the present context milk may be the lacteal secretion of any mammal. Thus, milk may be obtained by milking, e.g., cows, sheep, goats, buffaloes, or camels.

A dairy composition used in the method of the first aspect will generally have a reduced sodium content, i.e. it will comprise an amount of sodium which is lower than that conventionally present in a corresponding dairy composition.

All or part of the sodium conventionally present in the corresponding diary composition may be replaced by yeast extract which comprises at least 30% w/w 5′-ribonucleotides or at least 15% w/w of 5′-GMP plus 5′-IMP on the basis of sodium chloride free dry matter. Preferably the dairy composition which is used for preparing the dairy product with reduced sodium content comprises at least 25% w/w, 30% w/w, 35% w/w or 40% w/w less sodium, more preferably at least 45% w/w, 50% w/w, 55% w/w or 60% w/w less sodium than conventionally present in a corresponding diary composition used in the production of regular dairy products.

The dairy product with reduced sodium content produced with the method of the first aspect may be produced with any suitable process known in the art.

For example, the production of cheese will comprise a step in which curd is formed by enzymatic coagulation of a dairy composition with rennet, or by acidic coagulation with food grade acid or acid produced by lactic acid bacteria growth and it is subsequently separated from the whey. Depending on the type of cheese to be produced, the production of cheese may further comprise processing of the curd and aging of the resulting cheese. The yeast extract can be added to a diary composition at any stage of the cheese production process. Preferably the yeast extract will be added at a stage of the cheese production process wherein a homogeneous distribution of the yeast extract through the diary composition is possible. The yeast extract may e.g. be added to a composition comprising milk and/or one or more milk components and/or milk fractions which is the starting composition in the production of cheese, it may be added to an intermediate product in the production of cheese (e.g. curd or whey) or it may be added to a cheese product (e.g. it can be homogeneously mixed into a soft cheese). Preferably the yeast extract will be added to the curd. Some cheeses are periodically washed in a saltwater brine during their ripening. In this latter case the method of the invention may involve addition of the yeast extract to the surface of a diary composition wherein the diary composition is a ripening cheese. In another preferred embodiment, the yeast extract according to the present invention is distributed on top of a brined cheese, which is then vacuumised and packed in foil for (further) ripening.

The production of cheese with reduced sodium content will generally comprise in one or more steps of the production process the use of less sodium chloride and optionally of a lower amount of other sodium-containing salts then conventionally used in the same step of a production process for regular cheese.

In a preferred embodiment of the invention the method according to the first aspect further comprises adding a further salt (sodium chloride) substitute or further taste enhancer to the diary composition used in the production of a dairy product with reduced sodium content. Salt substitutes and salt taste enhancers known in the art, are e.g. LiCl, LiBr, KCl, NH₄Cl, other ammonium salts, MgCl₂ dipeptides and amino acids derivatives. The salt substitutes or further taste enhancers can be added to the diary composition in a way which is analogous to that described for the addition of the yeast extract.

In one embodiment, 0.001% to 2% w/w yeast extract on the total weight of dairy composition is used, preferably 0.01% to 2% w/w, more preferably 0.05% to 1% w/w yeast extract on the total weight of diary composition is used for preparing a dairy product with reduced sodium content.

In a second aspect the invention provides a dairy product with reduced sodium content, obtainable with the method of the first aspect, containing a yeast extract comprising at least 30% w/w 5′-ribonucleotides, on the basis of sodium chloride free dry matter. The dairy product with reduced sodium content of the second aspect has an improved salt perception if compared with the dairy product with reduced sodium content not comprising the yeast extract. Furthermore no bouillon or brothy note typical of the yeast extract can be perceived in the taste of the dairy of the second aspect. Also when the dairy product with reduced sodium content of the second aspect further comprises salt substitutes or salt enhancers mentioned above, the bitterness or off-flavour thereof is masked. In one embodiment the amount of yeast extract present in the dairy product with reduced sodium content is 0.01% to 2% w/w based on the dairy product weight, more preferably 0.05% to 1% w/w based on the dairy product weight.

In one embodiment the dairy product with a reduced sodium content in comparison to a corresponding dairy product, is prepared from a dairy composition, which dairy composition comprises (i) at least 30% w/w less sodium than the corresponding conventional dairy composition and (ii) 0.01% to 2% w/w of a yeast extract which comprises at least 30% w/w 5′-ribonucleotides on the basis of sodium chloride free dry matter.

In a third aspect the invention provides the use of a yeast extract comprising at least 30% w/w 5′-ribonucleotides, on the basis of sodium chloride free dry matter, to prepare a dairy product with reduced sodium content.

In a fourth aspect the invention provides the use of a yeast extract comprising at least 30% w/w 5′-ribonucleotides, on the basis of sodium chloride free dry matter, to improve the taste of a dairy product with reduced sodium content. In the context of the present invention “to improve the taste of a dairy product with reduced sodium content” means to improve the salt perception in the dairy product with reduced sodium content and optionally to mask the bitterness or off-flavour of further salt replacers (e.g. of potassium chloride) optionally present in the dairy product with reduced sodium content. Therefore, in another aspect the invention relates to a composition or blend suitable for preparing a reduced-sodium dairy product, wherein the blend comprises a yeast extract which comprises at least 30% w/w 5′-ribonucleotides (sfdm).

In one embodiment, the blend comprises a yeast extract which comprises at least 30% w/w 5′-ribonucleotides (sfdm) in combination with KCl, and preferably NH₄Cl. A suitable blend comprises 40-60% w/w, preferably 45-60% w/w KCl and 25-40% NH₄Cl. In a preferred embodiment, the blend comprises 45-60% w/w KCl, 25-40% NH₄Cl and 1-10% w/w of a yeast extract which comprises at least 30% w/w 5′-ribonucleotides (sfdm). More preferably, the blend comprises 51-55% w/w KCl; 30-37% w/w NH₄Cl; and 2-5% w/w of a yeast extract which comprises at least 30% w/w 5′-ribonucleotides (sfdm). The blend may further comprise 0-30% w/w, preferably 0-20% w/w, more preferably 0-10% w/w sucrose. The blend preferably comprises 10-25 parts by weight of KCl per part of a yeast extract which comprises at least 30% w/w 5′-ribonucleotides (sfdm). Preferably, the blend comprises 10-20 parts, more preferably 15 parts by weight of KCl per part of a yeast extract which comprises at least 30% w/w 5′-ribonucleotides (sfdm). In another embodiment, the blend comprises a yeast extract which comprises at least 30% w/w 5′-ribonucleotides (sfdm) in combination with melting salts, such as citrates and phosphates, in particular potassium citrate and potassium phosphates.

As mentioned before, the amount of blend to be used in the dairy product will depend on the type of dairy product produced. All the products and blends of the present invention may advantageously be used in a low-sodium, reduced-sodium or no-sodium diet or in the treatment of hypertension or cardiovascular disease.

Embodiments of the first aspect of the invention are also applicable to the other aspects of the invention.

### EXAMPLES

### Example 1

### The Effect of Yeast Extract and the Effect of the 5′-Ribonucleotide Concentration of Yeast Extract on the Taste of a Cheese with Reduced Sodium Content

This example was set up to demonstrate the effect of yeast extracts and of the nucleotide purity of the applied yeast extracts on the taste perception in cream cheese with reduced salt levels.

### Ingredients:

Yeast extract type 1—a 5′-ribonucleotide-rich yeast extract containing 11.2% w/w 5′GMP+5′IMP, 22.4% w/w 5′-ribonucleotides and 20% w/w of sodium chloride on dry matter (corresponding to 14% w/w 5′-GMP+5′-IMP, 28% w/w 5′ ribonucleotides on sodium chloride free dry matter) (DSM Food Specialties—The Netherlands)
Yeast extract type 2—a 5′-ribonucleotide-rich yeast extract containing 20% w/w 5′GMP+5′IMP and <1% w/w sodium chloride on dry matter (40% w/w 5′-ribonucleotides on dry matter) (DSM Food Specialties—The Netherlands)
Starter culture—DSM Delvo-Tec® BM 101 DSF (DSM Food Specialties—The Netherlands).
Locust bean gum—Viscogum® (Texturant Systems—Belgium)

The two different yeast extract types were used to prepare salt replacement blends, as listed in Table 1.

TABLE 1
salt blend compositions

| Ingredient | Blend A | Blend B | Blend C |
|---|---|---|---|
| Potassium chloride | 78.1 g | 79.3 g | 80.9 g |
| Ammonium chloride | 48.7 g | 49.5 g | 50.5 g |
| Sucrose | 48.7 g | 49.5 g | 50.5 g |
| Yeast extract type 1 | 6.5 g | — | — |
| Yeast extract type 2 | — | 3.6 g | — |
| Total blend | 182.0 g | 182.0 g | 182.0 g |

Cream cheese was prepared according to the procedure described below.

Reference cheese:

136 kg of whole milk with added cream to a butterfat level of 12% was pasteurized for 19 seconds at 77° C. and homogenized in a pressure homogenizer. The homogenized milk was cooled to about 26° C. and 136 g of starter culture was added. After thirteen hours, agitation was started and the temperature was increased to 80° C. Next, the mixture was pumped to the separator and the cream cheese was collected in a swept surface tank. 120 g of Locust Bean Gum and 364 g of salt (NaCl) were added. After 30 minutes, the mixture was homogenized and packed. Other cheeses were prepared in which part of the NaCl was omitted or replaced by one of the blends (see Table 2). The different cream cheeses were judged by a taste panel on the overall taste and special attention was paid to salt perception, brothy or bouillon notes and bitter notes. The taste results after 20 days of shelf life are presented in Table 2.

TABLE 2

| | NaCl (g) | Blend (g) | Taste panel results |
|---|---|---|---|
| Reference | 364 | — | |
| Test 1 | 182 | — | Less salty than reference |
| | | | and tests 2, 3 and 4 |
| Test 2 | 182 | 182 g of blend A | Salt perception comparable |
| | | | to reference, slight bouillon |
| | | | off-note |
| Test 3 | 182 | 182 g of blend B | Salt perception comparable |
| | | | to reference |
| Test 4 | 182 | 182 g of blend C | Less salty than test 2, |
| | | | slightly bitter |

The results demonstrate that the taste of cheese with and NH₄CL and KCl, but without nucleotide-rich yeast extract (test 4) is characterized by a bitter note. This is undoubtedly related to the presence of the potassium chloride in the salt replacement blend.

The results also clearly demonstrate that even though the salt perception of the sample comprising yeast extract 1 (test 2) is comparable to the reference, the overall taste of the cheese is characterised by a bouillon off-note, originating from the yeast extract

No bouillon note was perceived in test 3 where yeast extract 2 was applied. This clearly demonstrates the effect of the 5′-ribonucleotide containing yeast extract on the presence of bouillon off-notes in the application. Yeast extract type 2 is also able to mask the bitter off-note, due to the presence of potassium chloride. In addition, it enhances the salt perception, compared to test 4 (test without yeast extract).

### Example 2

### The Effect of a 5′-Ribonucleotide Containing Yeast Extract on the Taste of a Processed Cheese Product with Reduced Sodium Content

This example demonstrates the effect of a 5′-ribonucleotide containing yeast extracts on the taste perception in processed cheese with reduced salt levels.

Sample 1: a standard processed Cheddar cheese, prepared using sodium-containing melting salts.
Sample 2: a reduced sodium processed Cheddar cheese, in which the sodium-containing melting salts were replaced by potassium-containing melting salts during preparation.
Sample 3: a reduced sodium processed Cheddar cheese according to sample 2, to which 0.1% w/w of a 5′-ribonucleotide-rich yeast extract containing 20% w/w 5′GMP+5′IMP and <1% w/w sodium chloride on dry matter (40% w/w 5′-ribonucleotides on dry matter) (DSM Food Specialties—The Netherlands) was added during preparation.

The cheeses were tested by a taste panel (n=6) in a randomized blind experiment. Saltiness and cheese taste intensity were ranked on a scale from 1 (lowest) to 8 (highest). Mean scores are presented in Table 3.

TABLE 3

| Trial | Sample 1 | Sample 2 | Sample 3 |
|---|---|---|---|
| Saltiness | 3.0 | 2.0 | 7.3 |
| Intensity | 3.2 | 2.5 | 7.0 |

The test panel members were asked to indicate whether an off-flavour was noticed. For each processed cheese sample there was only 1 recordal of an off-flavour.

The results show that also in processed cheese products, the 5′-ribonucleotide containing yeast extract according to the invention can increase saltiness perception and enhance the taste intensity of the dairy product.

### Example 3

### Preparation of a Margarine (40% Fat) with Reduced Sodium Using Comprising 5′-Ribonucleotide Containing Yeast Extract

This example demonstrates how a margarine with reduced sodium content may be prepared using a 5′-ribonucleotide containing yeast extract.

In a standard low fat spread containing 40% fat, with a sodium level (NaCl) of 1.5%, the NaCl is replaced with 1.2% of NaCl and 0.015% of 5′-ribonucleotide-rich yeast extract containing 20% w/w 5′GMP+5′IMP and <1% w/w sodium chloride on dry matter (40% w/w 5′-ribonucleotides on dry matter) (DSM Food Specialties—The Netherlands). The prepared margarine will be comparable in taste to a corresponding margarine product which contains 1.5% NaCl (normal sodium levels).

### Example 4

### Preparation of a Margarine (40% Fat) with Reduced Sodium Using a Composition Comprising a 5′-Ribonucleotide Containing Yeast Extract

This example demonstrates how a margarine with reduced sodium content may be prepared using a composition comprising KCl, NH₄Cl, of a 5′-ribonucleotide containing yeast extract.

In a standard low fat spread containing 40% fat, with a sodium level (NaCl) of 1.5%, the NaCl is replaced with 1.0% of NaCl and 0.375% of a non-sodium salt replacer composition. The composition contains 51-55% w/w KCl, 30-37% w/w NH₄Cl, 1-10% w/w sucrose and 2-5% w/w 5′-ribonucleotide-rich yeast extract containing 20% w/w 5′GMP+5′IMP and <1% w/w sodium chloride on dry matter (40% w/w 5′-ribonucleotides on dry matter) (DSM Food Specialties—The Netherlands). The prepared margarine will be slightly more salty than a corresponding margarine which contains 1.5% NaCl (normal sodium levels) and will be high in creaminess and butter flavor.

1. A method for preparing a dairy product with reduced sodium content in comparison to a corresponding dairy product, wherein a yeast extract comprising at least 30% w/w 5′-ribonucleotides, on the basis of sodium chloride free dry matter, is added to a dairy composition used in the production of a dairy product with reduced sodium content in comparison to a corresponding dairy product.

2. Method of claim 1, wherein the yeast extract comprises at least 15% w/w of 5′-GMP plus 5′-IMP on the basis of sodium chloride free dry matter.

3. Method of claim 2 wherein the yeast extract contains at least 20% w/w 5′-GMP plus 5′-IMP on the basis of sodium chloride free dry matter.

4. Method of claim 1 wherein the yeast extract is prepared from Saccharomyces yeast.

5. Method of claim 1 wherein the dairy product is a cheese, a cheese analogue, a cream cheese, a butter, a spread, a margarine or a processed cheese.

6. Method according to claim 5, wherein the product is a processed cheese wherein part of the sodium-containing melting salts is replaced.

7. Method according to claim 1 wherein the method further comprises adding a further sodium chloride substitute or further taste enhancer to the dairy composition.

8. Method according to claim 1 wherein the dairy product with reduced sodium contains at least 25% w/w less sodium than conventionally present in the corresponding dairy product.

9. A dairy product with reduced sodium content in comparison to a corresponding dairy product, wherein the dairy product contains a yeast extract comprising at least 30% w/w 5′-ribonucleotides, on the basis of sodium chloride free dry matter.

10. Dairy product with a reduced sodium content according to claim 9, wherein the dairy product is prepared from a dairy composition, which dairy composition comprises (i) at least 30% w/w less sodium than the corresponding conventional dairy composition and (ii) 0.01% to 2% w/w of a yeast extract which comprises at least 30% w/w 5′-ribonucleotides on the basis of sodium chloride free dry matter.

11. Dairy product according to claim 9, wherein the dairy product is a cheese product, a cheese analogue, a cream cheese, a butter, a spread, a margarine or a processed cheese.

12. A composition for preparing a dairy product with a reduced sodium content in comparison to a corresponding dairy product, which composition comprises a yeast extract which comprises at least 30% w/w 5′-ribonucleotides on the basis of sodium chloride free dry matter.

13. Composition according to claim 12, which further comprises (a) 45-60% w/w KCl and (b) 25-40% w/w NH₄Cl.

14. Composition according to claim 12, which further comprises citrate or phosphates.

15. Composition according to claim 12-14, which further comprises 0-10% w/w sucrose.

16. Composition according to claim 12 which comprises 1-10 w/w % of a yeast extract which comprises at least 30% w/w 5′-ribonucleotides on the basis of sodium chloride free dry matter.

17. A yeast extract which comprises at least 30% w/w 5′-ribonucleotides on the basis of sodium chloride free dry matter, for use in a reduced-sodium, no-sodium or low-sodium diet.

18. A yeast extract which comprises at least 30% w/w 5′-ribonucleotides on the basis of sodium chloride free dry matter, for use in the treatment of hypertension or cardiovascular disease.

19. Use of a yeast extract comprising at least 30% w/w 5′-ribonucleotides, on the basis of sodium chloride free dry matter, for the preparation of a dairy product with reduced sodium content in comparison to a corresponding conventional dairy product.

20. Use of a yeast extract comprising at least 30% w/w 5′-ribonucleotides, on the basis of sodium chloride free dry matter, for taste enhancement or improvement in a dairy product with reduced sodium content.

21. Use of a yeast extract which comprises at least 30% w/w 5′-ribonucleotides on the basis of sodium chloride free dry matter, in a reduced sodium dairy product according to claim 9 in a reduced-sodium, no-sodium or low-sodium diet.

22. Use of a yeast extract which comprises at least 30% w/w 5′-ribonucleotides on the basis of sodium chloride free dry matter for the preparation of a food with reduced sodium in comparison to a corresponding food, which food can be used in a reduced-sodium, no-sodium or low-sodium diet.

23. Use of a yeast extract which comprises at least 30% w/w 5′-ribonucleotides on the basis of sodium chloride free dry matter for the preparation of a food with reduced sodium in comparison to a corresponding food, which food can be used in the treatment of hypertension or cardiovascular disease.

24. Use according to claim 22 wherein the food is a dairy product.
